(19) United States
(12) Patent Application Publication
(10) Pub. No.: US 2014/0065571 A1
(43) Pub. Date: Mar. 6, 2014

(54) INTRAORAL MAXRETRACTOR

(71) Applicant: CHEN WEN TSENG, Taipei City (TW)

(72) Inventor: CHEN WEN TSENG, Taipei City (TW)

(21) Appl. No.: 13/598,624

(22) Filed: Aug. 30, 2012

(51) Int. Cl. A61B 1/24

(52) U.S. Cl. 433/140

(57) ABSTRACT

An intraoral retractor includes a main body plane with one curved end and is dented in the middle to avoid injure the frenum; and the other end is a handle bended 30 degree away from the plan of the body. The grasper is inserted within a patient's mouth to retract the lip and open the patient's mouth vertically, while the handle allows easy operation. With the retractor, the operator can pull the patient's upper or lower lip as desired and examine the upper or lower dentition, which is highly useful for intraoral photography, dental checkups and treatments.

### BACKGROUND OF THE INVENTION

1. Field of the Invention

The invention relates to dental appliances, and, in particular, to a dental device for use when intraoral photography is required or when performing dental hygiene activities.

2. Description of the Prior Art

Healthy teeth serve as the primary checkpoint for maintaining a healthy body because teeth are used for chewing, ripping and tearing food apart, and grounding food to make it easier to digest in stomach. Without adequate chewing, a large piece of food can increase the workload of stomach and lead to digestive problems or even cause damage in stomach and affect health. The majority of human teeth is replaced once a lifetime and cannot be repaired; therefore, oral health is an important topic for everyone. In addition to personal hygiene, regular checkups or treatments are necessary in preventing dental caries. Sharp tools make good work. Teeth are positioned and usually enclosed in the oral cavity, and visualization of the whole dentition, even when mouth is wide opened, is difficult. Hence, it is required for a dentist to use other tools when treating patients. Nonetheless, most dental appliances are designed in accordance with the needs of a dentist instead of a patient's feeling. Thus, dental checkups are usually considered unpleasant experiences for many people. In spite of the encouragement received from the dentists or nurses, most people are anxious or stressed when seating in the clinics and listening to the noises made by various machines. The harsh smell of detergents in doctor's clinics often contributes to the unpleasant feeling of patients. Therefore, improvements of the dental appliances which make the tools more humane and with enhanced functions not only can reduce the stress in patients, but efficiently assist dentists in performing examination and/or treatment activities. With reference to a common dental tool shown in FIG. 7, a dental retractor (3) is used by a dentist to retract a patient's (2) lip (21) when examining the teeth (22), but the dental retractor (3) can only retract the lip to certain extent, and usually two or more retractors (3) are needed to allow access to the hard to reach areas of a patient. The restricted working space requires a precisely coordinated interplay of the movements of the dentist performing the treatment and may cause discomfort in patients. Likewise, the dentist has to be extremely carefully to prevent accidently injury to the patients.

Based on the abovementioned shortcomings of the conventional devices, the inventor(s) of the present invention, after years of meticulous research, developed the intraoral MaxRetractor to overcome these problems.

### SUMMARY OF THE INVENTION

The main purpose of the present invention is to provide an intraoral retractor for used by a dentist in dental examination and/or operation to allow easy pulling of a dental patient's upper or lower lip and to improve the quality of dental care.

Another aspect of the invention involves a mouth-fitted and sheet-like intraoral retractor without multiple spikes, and said retractor can be inserted within a patient's mouth and reduce the rejection from patients.

A further aspect of the present invention is to provide a low-cost intraoral retractor with simple structure for easy promotion and create a win-win situation.

To achieve the foregoing aims, the intraoral MaxRetractor of the present invention comprises a body plane with one curved end (a grasper) and a handle at the other end. The grasper is used to retract a patient's lip upward or downward and the handle is for easy holding by the operator for examination of the whole dentition. The grasper is designed as curve-shaped to accommodate the shape of the lips; moreover, the width of the grasper is less than that of a mouth for ergonomic purposes. Hence, patient's teeth can be easily examined or treated when using a MaxRetractor to pull and lift the lip.

### BRIEF DESCRIPTION OF THE DRAWING

The drawings illustrate both the design and utility of preferred embodiments of the present invention, in which similar elements are referred to by common reference numbers, wherein:

FIG. 1 is a perspective view of a MaxRetractor according to the present invention;

FIG. 2 is a front perspective view of the MaxRetractor;

FIG. 3 is a side perspective view of the MaxRetractor;

FIG. 4 is a schematic illustration of operation of the MaxRetractor;

FIG. 5 is a side perspective view of operation of the MaxRetractor (A);

FIG. 6 is a side perspective view of operation of the MaxRetractor (B);

FIG. 7 is a schematic illustration of operation of a common dental retractor.

### DETAILED DESCRIPTION OF THE PREFERRED EMBODIMENT

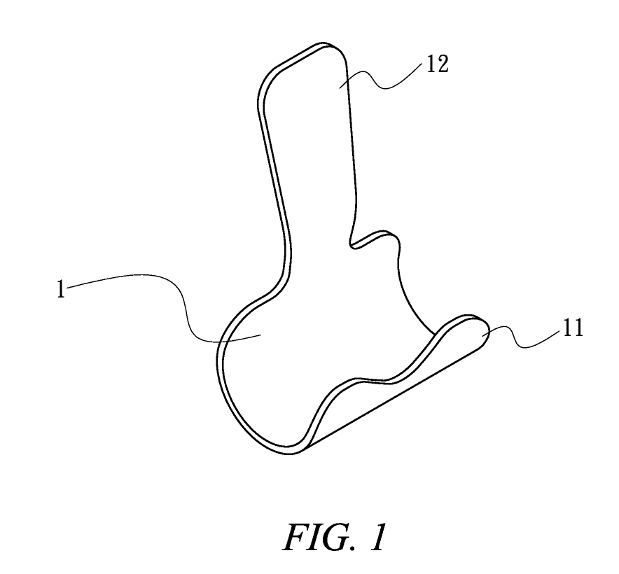

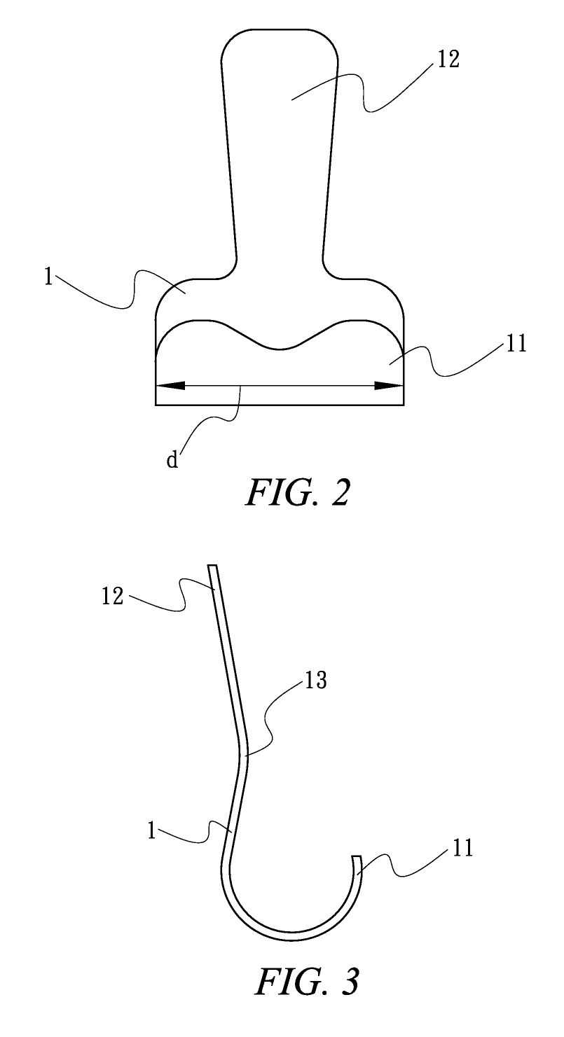

With reference to FIGS. 1-3, the present invention provides an intraoral MaxRetractor comprises a body plane 1 with a curved end (a grasper) 11 and a handle 12 at the other end.

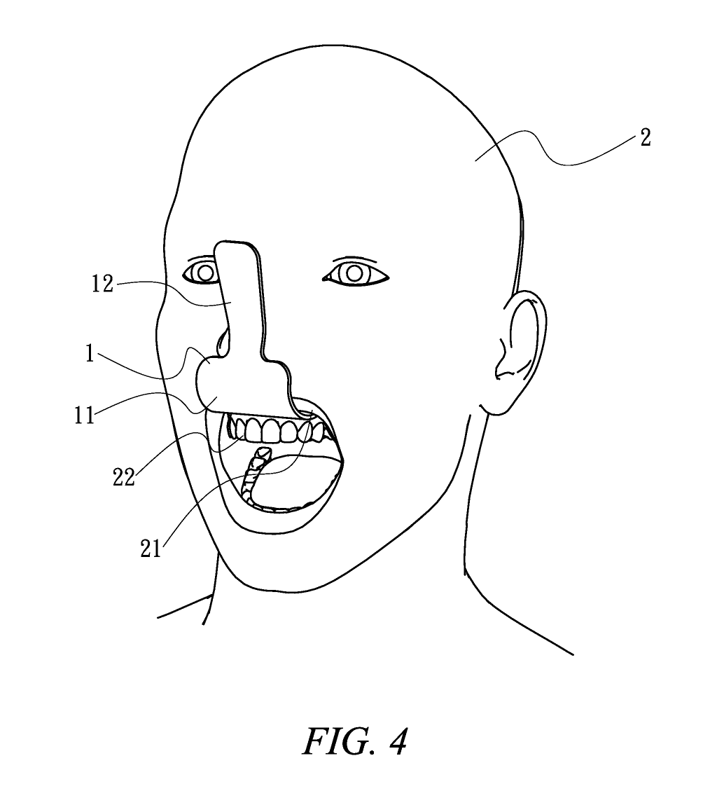

As shown in FIG. 4, said grasper 11 is designed for retracting and pulling the patients' lip 21 for easy access to the patients' mouth for examination and/or treatments. The handle 1 is easy to hold and allows the operator (the dentist) to retract and pull the lip.

Therefore, the operator can easily pull the patient's lip 21 and observe the entire tooth 22 for easy examination and/or treatments. Additionally, for ergonomic purposes, the “d” section of the grasper 11 can be bended to accommodate the shape of a patient's lip and to reduce discomfort in the patient. The width of the section “d” is less than the width of said patient's mouth which allows easy deposition of the retractor between lip 21 and teeth 22 within said patient's 2 mouth and effortlessly retract and lift the patient's lip 21.

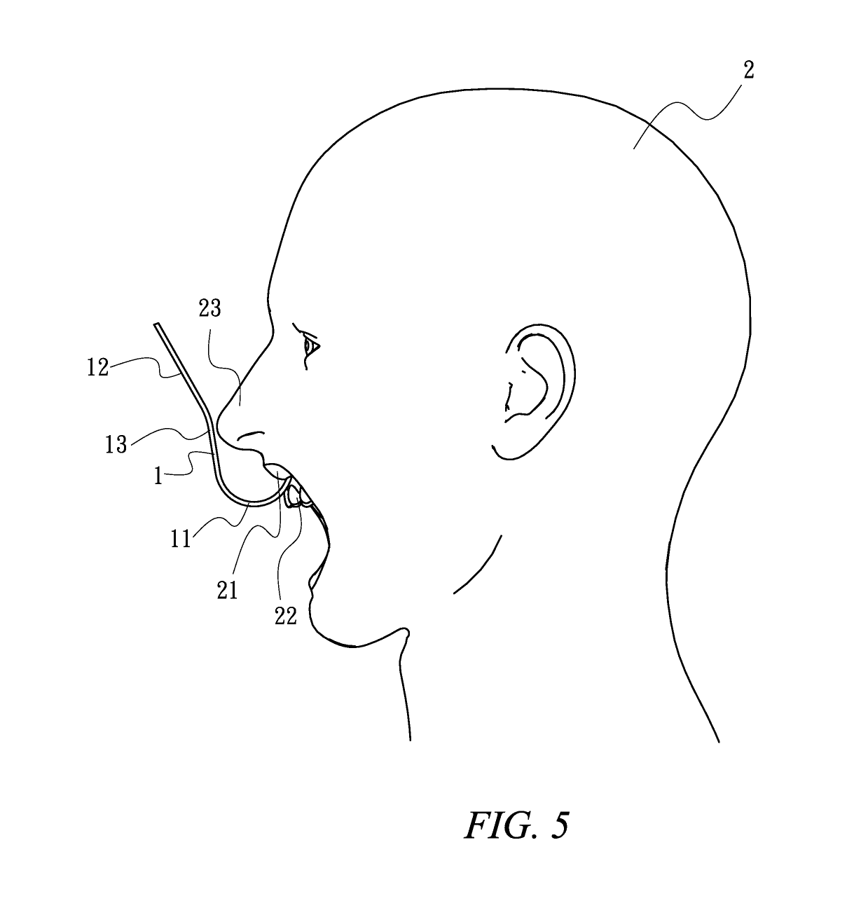

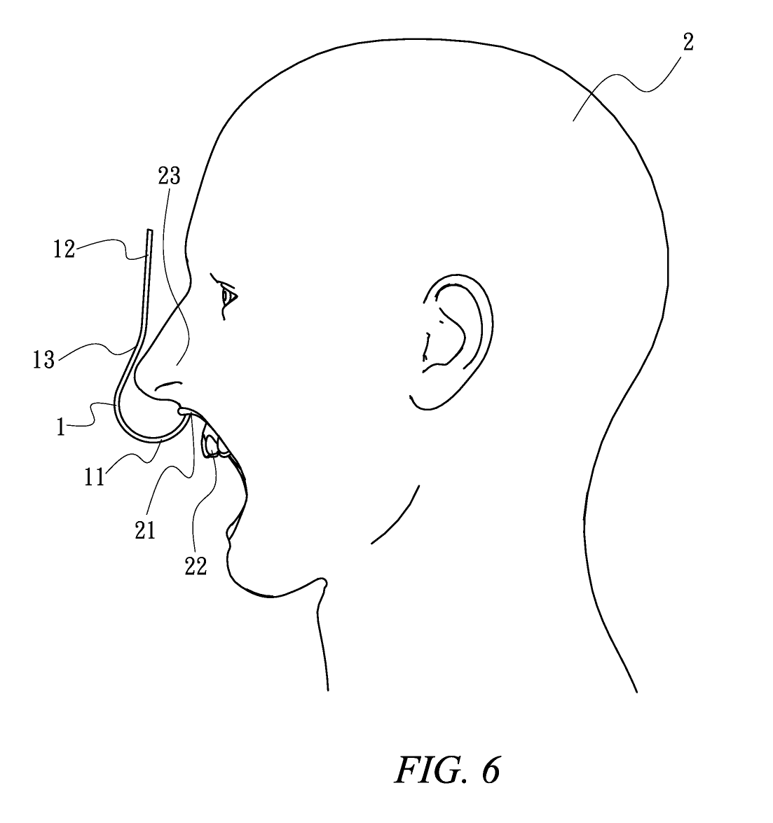

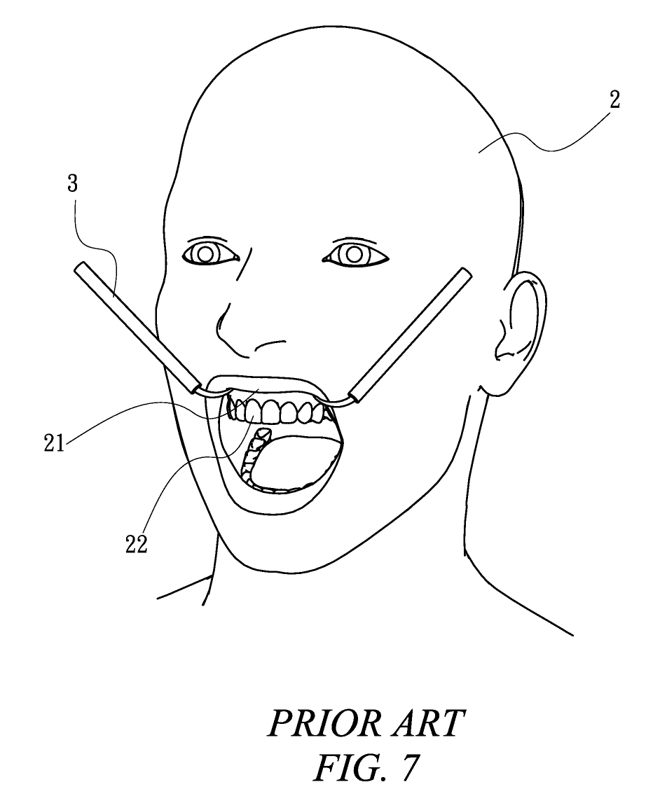

With reference to FIG. 5 and FIG. 6, the handle 12 is designed with a curved end 13 in the opposite direction of the grasper 11. When retracting the lip 21 with the grasper 11, the handle 12 will not touch the patient's 2 nose 23 because the curved end 13 design of the handle, and consequently allows painless lifting and examination and/or treatment.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. An intraoral retractor, comprising: a main body having one bended end that serves as a grasper for retracting and opening a patient's mouth and a handle formed at the other end opposite to said bended end to allow effortless operation, wherein said grasper is formed with a curved flange that extends along a widthwise direction of said grasper and that is formed with a notch portion so as to allow access to the patient's mouth and to retract the patient's lip.

2. (canceled)

3. (canceled)

4. The intraoral retractor of claim 1, wherein said handle has one end that is distal to said grasper and that is bended in a reverse direction of said grasper, which allows retracting of the patient's lip with said grasper without touching the patient's nose.
